(19) United States
(12) Patent Application Publication
(10) Pub. No.: US 2024/0226794 A9
(43) Pub. Date: Jul. 11, 2024

(54) PLANT AND PROCESS FOR REDUCTION OF THE CARBON DIOXIDE CONTENT OF ATMOSPHERIC AIR

(71) Applicant: OBRIST TECHNOLOGIES GMBH, Lustenau (AT)

(72) Inventors: Frank Obrist, Bregenz (AT); Robert Schlögl, Berlin (DE); Simon Moll, Niklasdorf (AT)

(21) Appl. No.: 18/277,920

(22) PCT Filed: Jan. 13, 2022
(86) PCT No.: PCT/EP2022/050633

§ 371 Date: Aug. 18, 2023

(30) Foreign Application Priority Data
Feb. 26, 2021 (DE) 10 2021 104 746.3

(51) Int. Cl. B01D 53/02; C01B 5/00; C01B 32/05; C07C 29/151; C25B 1/04; C25B 15/08

(52) U.S. Cl. CPC B01D 53/02; C01B 5/00; C01B 32/05; C07C 29/1518; C25B 1/04; C25B 15/081; B01D 2257/504; B01D 2258/06

(57) ABSTRACT

The disclosure relates to a plant, especially power plant, for reduction of the carbon dioxide content in atmospheric air, especially for improvement of atmospheric air quality. The plant has at least one electrolysis unit for oxygen production, at least one carbonization unit for carbon synthesis, especially a Bosch reaction unit, and at least one unit for cleaning of ambient air from an outside atmosphere surrounding the plant. The carbonization unit synthesizes carbon from carbon dioxide which is obtained from the atmosphere by means of the carbon dioxide sorption unit and this carbon is stored, in order to effectively reduce the proportion of carbon dioxide in the atmosphere. The disclosure further relates to a method of operating such a plant, with which the carbon dioxide content in the atmosphere can be efficiently reduced.

The invention relates to a plant for reduction of the carbon dioxide content of atmospheric air, in particular for reduction of the carbon dioxide content of atmospheric air and of water, preferably sea water. The invention further relates to a method for operating such a plant.

Since the beginning of the industrial revolution in 1800, the atmospheric CO₂ of previously stable 280 ppmv (parts per million by volume) has risen to 410 ppmv in 2020. It is predicted that this rise will continue or respectively further intensify if no techniques are used for curbing the carbon emissions.

The ratified Paris Agreement names as its main aim keeping the rise of the global average temperature to below 2° C. above the preindustrial level, which requires a reduction of CO₂ emissions to zero by 2050. Proposals for limiting these emissions include the use of biofuels, solar energy and wind turbines. The reduction of the hitherto CO₂ emissions, therefore the limiting of the rise of the CO₂ content in the atmosphere, is not, however, sufficient in the long term, in order to correct the disproportion of oxygen and CO₂ in the atmosphere which has arisen hitherto through an overproduction of CO₂. Rather, a need exists not only to not further increase the CO₂ content in the atmosphere in the long term, but rather to actively reduce it.

The natural carbon cycle has occurred over a long time in such a way that CO₂ is present in a particular amount in the atmosphere. Plants play a major role here, which sorb the carbon from the CO₂ through photosynthesis and emit the oxygen content to the atmosphere again. The CO₂ is thereby removed from the air (over 100 billion metric tons of carbon are received by the plants annually in this way). It is generally known that growing forest, particular aged between 10 and 40 years, is very well suited to absorb the carbon from the CO₂ located in the air, and to emit the oxygen to the atmosphere. Usually, such a forest on an area of one hectare emits ca. 15 to 30 metric tons of oxygen to the atmosphere per year. The emission quantity of oxygen depends here on the type of forest (deciduous forest, coniferous forest or mixed forest).

The natural forest has the disadvantage that the effective CO₂ absorption or respectively oxygen production is limited to the above-mentioned age period. The dependence of the photosynthesis process on sunlight constitutes a further restriction. Whereas the forest can absorb CO₂ and thus produce oxygen in daylight, this is not possible at night. Furthermore, after the rotting or felling of trees, new trees must be planted again, in order to maintain the natural CO₂ cycle. This entails a great effort.

As forest land has decreased drastically in the past decades and is continually declining, it is essential to develop new technologies which can be implemented in a short period of time and are able to support the still existing natural forest in its function and to not only slow down global warming, but contribute to actively reversing at least in part the global warming brought about through industrialization.

The invention is therefore based on the problem of indicating a plant which through a continuous process supports the natural forest in its function and thereby not only slows down global warming, but in the long term reverses it at least in part. The invention is further based on the problem of indicating a method for operating such a plant.

According to the invention, this problem is solved with regard to the plant by the subject of Claim 1. With regard to the method, the above-mentioned problem is solved by the subject of Claim 17.

In practice, the problem is solved by a plant, in particular a power plant, for reduction of the carbon dioxide content in atmospheric air, in particular for reduction of the carbon dioxide content in atmospheric air and proportionally in water, preferably sea water, wherein the plant comprises the following:
at least one electrolysis unit for oxygen production, which is connected to at least one water supply line to receive a water quantity, and is adapted to decompose a received water quantity into an oxygen partial quantity and a hydrogen partial quantity by electrolysis;
at least one hydrogen transport device, which connects the electrolysis unit to a carbonization unit for carbon synthesis, in particular a Bosch reaction unit;
at least one carbon dioxide sorption unit for cleaning ambient air of an outside atmosphere surrounding the plant, which has at least one air inlet for the supply of the ambient air and at least one downstream sorber device, which is adapted to extract a carbon dioxide quantity from the ambient air; and
at least one carbon dioxide transport device, which connects the carbon dioxide sorption unit to the carbonation unit.

The electrolysis unit has at least one oxygen outlet for the emission of the oxygen partial quantity, and the carbon dioxide sorption unit has at least one air outlet for the emission of cleaned ambient air, wherein the oxygen outlet and the air outlet open into the outside atmosphere. The carbonation unit has a carbon outlet for the removal of carbon.

The plant has, furthermore, at least one power generating unit for the self-sufficient power supply of the plant, wherein the power generating unit uses, generating power, one or more, in particular exclusively, regenerative energy sources.

In a preferred embodiment of the plant, the power generating unit is at least one photovoltaic unit for the conversion of solar energy into power. The use of a photovoltaic unit is particularly preferred, as the energy generating costs are particularly low here.

Compared to other technologies for regenerative energy generation, the energy generation by means of photovoltaics is three to ten times more economical. This applies in particular if the plant is constructed in a region with a high duration of hours of sunshine, for example in Saudi Arabia.

Although an energy generation by photovoltaics is preferred, the power generating unit can have, additionally or alternatively, at least one wind power unit for the conversion of wind energy into power. The wind power unit can comprise one or more wind turbines. Additionally or alternatively, the power generating unit can comprise at least one water power unit for the conversion of water energy into power. This water power unit can be at least one hydropower plant, in particular river power station or pumped storage power station. The water power unit can comprise additionally or alternatively a wave power plant. Furthermore, additionally or alternatively the power generating unit can be a thermal unit for the conversion of thermal energy into power. The thermal unit can be adapted to convert heat from at least one earth layer lying under the earth surface into power. Other thermal units are possible.

The plant can have, furthermore, at least one buffer store for storing energy. For example, the buffer store can be adapted to store electric power. Alternatively, the buffer store can be adapted to store hydrogen. The latter is particularly preferred. Through the buffer store, the energy supply of the plant is also enabled in the night, so that the plant can be operated without operational interruption. The plant and the method can thus be operated continuously.

The invention has various advantages. In order to produce oxygen for emission into the outside atmosphere, only water is necessary as base material for the plant, which is decomposed into its components oxygen and hydrogen by an electrolysis process. This process is designated as water electrolysis. The electrolysis unit is connected to the water supply line to receive a quantity of water for the electrolysis process. The quantity of water can be a quantity of fresh water or a quantity of desalinated sea water. Furthermore, at least one processing unit, in particular a desalination unit, can be provided, which processes the quantity of water before the electrolysis process, in particular cleans it and/or desalinates it.

When the received quantity of water is divided by the electrolysis unit into an oxygen partial quantity and a hydrogen partial quantity, the separated oxygen partial quantity is discharged through the oxygen outlet of the electrolysis unit into the outside atmosphere. Thereby the air of the outside atmosphere is mixed with fresh oxygen and the natural forest is supported in the oxygen production.

The oxygen outlet can be formed by at least one line, in particular a pipeline, which extends from the electrolysis unit to the outside atmosphere. Alternatively, the oxygen outlet can be formed by a chimney through which the separated oxygen partial quantity is able to be discharged into the atmosphere. For discharging the oxygen partial quantity, at least one ventilator, in particular a blower, can be arranged between the electrolysis unit and the oxygen outlet.

The separated hydrogen partial quantity is directed by means of the hydrogen transport device at least partially to the carbonation unit. The hydrogen transport device can be a pipeline which is connected to the electrolysis unit and to the carbonation unit. The plant can have an intermediate hydrogen store, which intermediately stores the hydrogen partial quantity before delivery to the carbonation unit. The hydrogen transport device then preferably connects the electrolysis unit to the intermediate hydrogen store and the latter in turn to the carbonation unit. The intermediate hydrogen store can be a container, in particular a pressure container.

The carbon dioxide sorption unit is adapted to extract a carbon dioxide quantity from the ambient air. The carbon dioxide sorption unit therefore serves for cleaning the ambient air of the outside atmosphere of carbon dioxide. For this, the carbon dioxide sorption unit has the sorber device, which is adapted to remove at least a carbon dioxide quantity from the ambient air. The sorber device is preferably an amine exchanger. Other sorber facilities for the extraction of carbon dioxide from air are possible.

The carbon dioxide sorption unit has the advantage that the CO concentration in the atmosphere is reduced and thus is brought closer again to the original concentration before industrialization. This constitutes a partial function of the natural forest, so that the latter is further supported. Advantageously, global warming is thereby slowed down.

Through the carbon dioxide transport device, the extracted carbon dioxide quantity is directed to the carbonation unit. The carbon dioxide transport device can be a pipeline which is connected to the carbon dioxide sorption unit and to the carbonation unit. The plant can have an intermediate carbon dioxide store, in which the carbon dioxide quantity is intermediately stored by the carbon dioxide transport device before passing on to the carbonation unit. The carbon dioxide transport device can connect the carbon dioxide sorption unit to the carbon dioxide intermediate store and to the carbonation unit. The carbon dioxide intermediate store can be a container, in particular a pressure container.

The carbonation unit processes to water and carbon the hydrogen partial quantity, delivered by the hydrogen transport device, and the carbon dioxide quantity delivered by the carbon dioxide transport device. Preferably a Bosch reaction is used for this. Other carbonation methods are also possible. For example, the carbonation unit can be configured to carry out a Kvaerner process or a CO₂ plasma burner method. The carbon dioxide of the carbon dioxide quantity is therefore split into carbon and oxygen, wherein the oxygen connects together with the hydrogen of the hydrogen partial quantity to water. The carbon can be removed via the carbon outlet of the carbonation unit, for example for a further processing or storage. In this way, the carbon dioxide content in atmospheric air is efficiently reduced.

The plant which is described here forms a means by which the carbon dioxide content of the atmospheric air can be reduced. In other words, an undesired reduction of the oxygen content is prevented by the plant by the CO₂ content in the air being reduced. By the plant according to the invention a quantity regulation of the components in the atmospheric air is thus made possible, so that an existing imbalance of quantities of the air components can be equalized.

The invention has the further advantage that the plant is able to be operated continuously irrespectively of daytime and nighttime. Unlike natural forest, which requires sunlight for photosynthesis, carbon dioxide is able to be removed continually from the atmosphere and oxygen is able to be delivered continuously to the atmosphere by the plant. Furthermore, the oxygen emission performance and carbon dioxide removal performance of the plant is substantially independent of a lifespan of the plant. Through the operation of the plant in a continuous process, oxygen is able to be produced and carbon dioxide is able to be sorbed and able to be removed from the atmosphere by carbonation in the long term. Thereby, the natural forest is not only reliably supported, but its function is in addition surpassed, as the storage of the carbon which is removed from the atmosphere takes place in the long term and the risk is reduced that the carbon is released again by burning for example of forest areas. In this respect it is advantageous if the carbon which is generated in the carbonation unit is delivered to a carbon store. The carbon store can be, in particular, a sea or respectively a seabed. In other words, the carbon, in particular in the form of graphite, can be stored permanently on the seabed.

Particularly preferably, the plant according to the invention is able to be operated as a large power plant in coastal regions, in particular with access to a body or water or the sea, as water is available for oxygen production in very large quantities. Preferably, the plant is configured for operation in very dry areas, in particular deserts. This has the advantage that such areas in which no or only minimal vegetation prevails are improved by appropriate use. The plant according to the invention forms substantially an artificial forest which undertakes a function of the natural forest and/or which supports the natural forest in its function. Furthermore, the plant can be operated in combination with a photovoltaic plant in an entirely energy-autonomous manner, i.e. without the use of fossil fuels.

In a preferred embodiment of the invention, the hydrogen transport device and the carbon dioxide transport device can be additionally connected to a methanol synthesis unit for the production of methanol, wherein the methanol synthesis unit has a methanol outlet for the removal of methanol. The plant can thus also be used in order to produce a CO₂-neutral fuel, namely methanol produced in a CO₂-neutral manner. This applies in particular when the energy supply of the plant takes place exclusively by regenerative energies, in particular a photovoltaic unit.

In the coming years, the main focus of climate protection will lie inter alia on maintaining the mobility of people as much as possible in a climate-neutral, in particular CO₂-neutral manner. Here, the regeneratively produced methanol is a significant energy carrier which can replace previous fossil fuels. In this respect, the plant can be initially used principally or entirely for the production of methanol, in order to meet the initially high demand. As soon as the demand decreases, because for example the climate-neutral mobility has been achieved comprehensively or mobility, particularly in the course of digitalization, loses importance, the plant can be operated so that successively the proportion of the methanol production is reduced and the proportion of carbonation and carbon storage is increased. Thus, in 2035 the plant could be operated for example so that it yields at 20% graphite for storage and 80% methanol, whereas the same plant produces in 2050 50% graphite for storage and 50% methanol and in 2070 90% graphite for storage and 10% methanol.

As in the carbonation in the carbonation unit water, in particular pure water, is produced, the efficiency of the plant can be advantageously increased, if at least a portion of the water is directed back to the electrolysis unit and is thus available for the production of hydrogen and oxygen. In this respect, in a preferred embodiment of the plant, provision is made that the carbonation unit is connected to the electrolysis unit by means of a water transport device.

The carbonation unit, in particular the carbon outlet, is preferably connected to a carbon store by means of a carbon transport device. This enables the long-term storage of the carbon and thus brings about the desired effect of reducing the carbon content in the atmosphere and thus at least partially revising the disproportion of carbon dioxide and oxygen which arose hitherto through industrialization.

The carbon transport device can be formed at least in part by a water return line. The water return line can open out in particular in a water reservoir, preferably the sea. The carbon or respectively the graphite can thus be stored in the long term on the seabed. It is indeed also possible to trade the carbon as active carbon. The overriding aim of the invention of reducing the carbon content in the atmosphere in the long term is, however, reliably achieved when the carbon is stored permanently. The seabed is suitable here particularly as a carbon store.

In this respect, it is preferred if the water return line is resistant to salt water. Likewise, the water supply line can be resistant to salt water. In particular, the water supply line and the water return line can open into a water reservoir, in particular a sea, in order to receive salt water from the water reservoir or to return it into the water reservoir. When the carbon transport device is formed at least in part by the water return line, the carbon can also be directed into the water reservoir via the water return line.

The water return line can have a desalination device, in order to desalinate the seawater before direction to the electrolysis unit.

The carbonation unit preferably has a catalyst which comprises iron, cobalt, nickel and/or ruthenium. This applies in particular when the carbonation unit is configured as a Bosch reaction unit.

It is further advantageous if in addition to the extraction of carbon dioxide from the ambient air, at least one carbon dioxide extraction unit is provided which is connected to the water supply line for the extraction of carbon dioxide from the quantity of water. In the water, in particular seawater, a considerable proportion of carbon is contained in the form of CO₂, which can be stored in this way in the long term in the form of graphite. The reduction of the carbon dioxide from the seawater likewise has an effect on the CO₂ content in the atmosphere, as through the reduction of the CO₂ content in the seawater, less CO₂ outgasses and arrives into the atmosphere.

In a preferred embodiment of the invention, the electrolysis unit has an output rate of an oxygen partial quantity per year of at least 700000 metric tons and/or the carbon dioxide sorption unit has an extraction rate of a carbon dioxide quantity per year of at least 400000 metric tons, in particular 600000 metric tons, in particular 640000 metric tons. The electrolysis unit can be adapted to separate from a water quantity of at least 1.5 kg, in particular of at least 1.7 kg, an oxygen partial quantity of at least 1.2 kg, in particular of at least 1.5 kg, and/or a hydrogen partial quantity of at least 0.1 kg, in particular of at least 0.15 kg.

The carbon dioxide sorption unit can be adapted to extract from an ambient air quantity of at least 3300 kg a carbon dioxide quantity of at least 1.1 kg, in particular at least 1.3 kg, preferably 1.375 kg.

A secondary aspect of the invention concerns a plant, in particular a power plant, for the use of the carbon dioxide content in atmospheric air, in particular for the use of the carbon dioxide content in atmospheric air and in water, preferably sea water, for the production of a liquid fuel, wherein the plant comprises the following:
at least one electrolysis unit for oxygen production, which is connected to at least one water supply line to receive a water quantity and is adapted to decompose a received water quantity by electrolysis into an oxygen partial quantity and a hydrogen partial quantity;
at least one hydrogen transport device, which connects the electrolysis unit to a methanol synthesis unit for the production of methanol;
at least one carbon dioxide extraction unit, which is connected to the water supply line for the extraction of carbon dioxide from the water quantity;
at least one carbon dioxide sorption unit for cleaning ambient air of an outside atmosphere surrounding the plant, which has at least one air inlet for the supply of the ambient air and at least one downstream sorber device which is adapted to extract a carbon dioxide quantity from the ambient air; and
at least one carbon dioxide transport device, which connects the carbon dioxide sorption unit to the methanol synthesis unit,

wherein the electrolysis unit has at least one oxygen outlet for the emission of the oxygen partial quantity, and the carbon dioxide sorption unit has at least an air outlet for the emission of cleaned ambient air, wherein the oxygen outlet and the air outlet open into the outside atmosphere, and wherein the methanol synthesis unit has a methanol outlet for the removal of methanol.

The plant has, furthermore, at least one power generating unit for the self-sufficient power supply of the plant, wherein the power generating unit uses one or more, in particular exclusively, regenerative energy sources for the power generation.

With this plant, the advantage exists that not only is the carbon dioxide from the atmosphere used, but also the carbon dioxide bound in the water, in order to produce methanol as a climate-neutral liquid fuel. This increases the raw material sources available for methanol production and in this respect offers a fail-safety. At the same time, the aim of reducing the carbon dioxide content in the atmosphere is further pursued. The further developments and advantages explained above with reference to the plant for the reduction of the carbon dioxide content in atmospheric air apply in this respect accordingly also to the plant for the production of liquid fuel.

A further coordinate aspect of the invention concerns a method for reduction of the carbon dioxide content in atmospheric air, in particular for the improvement of atmospheric air quality, in particular for operating a previously described plant, wherein in the method
a quantity of water is received by at least one electrolysis unit for oxygen production through at least one water supply line, and the received water quantity is decomposed into an oxygen partial quantity and a hydrogen partial quantity by electrolysis;
the hydrogen partial quantity is directed by at least one hydrogen transport device at least partially to a carbonation unit;
ambient air of an outside atmosphere surrounding the plant is cleaned by at least one carbon dioxide sorption unit, wherein the ambient air is delivered through at least one air inlet to a downstream sorber device and subsequently a carbon dioxide quantity is extracted from the delivered ambient air by the sorber device; and
the carbon dioxide quantity is directed by at least one carbon dioxide transport device to the carbonation unit.

In the method according to the invention, the oxygen partial quantity and the cleaned ambient air are emitted to the outside atmosphere, and the hydrogen partial quantity and the carbon dioxide quantity are converted in the carbonation unit, preferably a Bosch reaction unit, to water, carbon and heat. Thereby, a reduction of the carbon dioxide content in the atmospheric air and thus the equalization of an existing imbalance of the quantities of the air components is made possible.

In the method, the plant is supplied with power in a self-sufficient manner from one or more, in particular exclusively, regenerative energy sources.

Preferred further developments and advantages of the method will emerge from the subclaims and from the further developments and advantages disclosed in connection with the plants described above.

Thus, in a preferred embodiment of the invention, the Bosch reaction unit can be connected to the electrolysis unit by a water return device. In particular, the water occurring in the carbonation unit can be directed from the carbonation unit to the electrolysis unit and used therein for the production of hydrogen. The efficiency of the method according to the invention is thereby increased, as the proportion of fresh water which must be delivered to the process for the electrolysis is reduced.

It is advantageous if the carbonation unit, in particular the carbon outlet, is connected to a carbon store by means of a carbon transport device. The carbon which is produced in the carbonation unit can thus be delivered to the carbon store for long-term storage. The carbon store can be, in particular, a natural store, for example a seabed. In practice, the carbon removed from the carbonation unit, which can be solidified rock-like manner (graphite), can be stored in the long term in the sea. However, it is also possible to provide at least a portion of the carbon for industrial further processing, for example for the production of carbon fibres. The latter is, however, not primarily intended, because through the further processing and if applicable later burning of further processed products, no reduction of the carbon dioxide content in the atmosphere is achieved. An acceptable form of further processing consists in using the graphite as fertilizer or respectively for soil improvement in agriculture. As energy is required for the further transport of the graphite into the corresponding regions, which mostly leads to a discharge of carbon dioxide into the atmosphere, however, the efficiency declines with regard to the reduction of the carbon dioxide content in the atmosphere.

The heat arising during the carbonation in the carbonation unit, in particular in a Bosch reaction, can be directed to the carbon dioxide sorption unit and used there as energy for the carbon sorption. In this way, the efficiency of the entire method is further increased and the primary energy requirement of the plant or respectively of the method is reduced.

Preferably, the process temperature for the preferred Bosch reaction used for the carbonation, which takes place for the production of carbon from hydrogen and carbon dioxide in the carbonation unit, configured as a Bosch reaction unit, is between 530° C. and 730° C.

In a preferred embodiment, the electrolysis unit has an output rate of an oxygen partial quantity per year of at least 700000 metric tons. Preferably, the electrolysis unit is adapted to produce from a water quantity of at least 500000 metric tons, in particular of at least 700000, in particular 750000 metric tons, at least 700000 metric tons oxygen per year. Compared to natural forest, which has an annual oxygen output rate of 15 to 30 metric tons per hectare, the plant in this embodiment and with an assumed area by way of example of ca. 12 square kilometres, produces 5 times to 40 times more oxygen per year.

Alternatively or additionally, the carbon dioxide sorption unit preferably has an extraction rate of a carbon dioxide quantity per year of at least 400000 metric tons, in particular 600000 metric tons. Preferably, the carbon dioxide sorption unit is adapted to separate from an air quantity of 1450 to 1600 megatonnes, in particular of 1570 megatonnes, at least 400000 metric tons, in particular 600000 metric tons, in particular 640000 metric tons, carbon dioxide per year. Thereby, the CO₂ concentration is reduced by a continuous process in the air in considerable quantities.

In a further preferred embodiment, the electrolysis unit is adapted to separate from a water quantity of at least 1.5 kg, in particular of at least 1.7 kg, an oxygen partial quantity of at least 1.2 kg, in particular of at least 1.5 kg, and/or a hydrogen partial quantity of at least 0.1 kg, in particular of at least 0.15 kg. Preferably, the electrolysis unit is adapted to separate from a water quantity of 1.7 kg, an oxygen partial quantity of at least 1.4 kg, in particular at least 1.45 kg, preferably 1.5 kg, and a hydrogen partial quantity of at least 0.18 kg, preferably 1.1875 kg. It is advantageous here that the electrolysis unit is designed in a highly efficient manner and very large quantities of oxygen and hydrogen are produced.

In a further preferred embodiment, the carbon dioxide sorption unit is adapted to extract from an ambient air quantity of at least 3300 kg a carbon dioxide quantity of at least 1.1 kg to 2 kg, in particular at least 1.3 kg, preferably 1.375 kg. Thereby, the considerable reduction of the CO₂ concentration in the air is made possible.

Preferably, the electrolysis unit and/or the carbon dioxide sorption unit have respectively at least one mounting region, which is connectable or connected to a base, in particular of a building and/or structure. The electrolysis unit and/or the carbon dioxide sorption unit are preferably securely connected to the base by the mounting regions. Alternatively, the respective unit can be connected respectively to a separate base.

In the configuration of the plant as a large power plant, the electrolysis unit and/or the carbon dioxide sorption unit are formed on a large scale. The electrolysis unit and/or the carbon dioxide sorption unit can be arranged respectively in a separate operations building. The electrolysis unit and/or the carbon dioxide sorption can be arranged in separate operations buildings which adjoin one another directly or indirectly. Alternatively, the electrolysis unit and/or the carbon dioxide sorption unit can be arranged respectively together in an operations building. A combination of separate arrangement and shared arrangement of the respective electrolysis unit and/or carbon dioxide sorption unit is possible.

As a whole, the plant and the method carried out therewith are preferably designed so that annually at least 50,000 metric tons, in particular at least 100,000 metric tons, in particular at least 150,000 metric tons, in particular at least 200,000 metric tons, in particular at least 250,000 metric tons, graphite can be produced.

The plant can have its own infrastructure. For example, the plant can comprise at least one access road. Furthermore, the plant can consist of several structures. These can be industrial operations buildings, for example. In addition, it is possible that the plant comprises a harbour for ships. Furthermore, power lines can be provided, in order to supply the plant with power, for example from a photovoltaic unit.

In the configuration as a small power plant, the plant can be arranged in at least one housing. The housing can envelop the plant. The housing can be formed from plastic and/or metal. It is advantageous here that the plant can be used in communal buildings as part of a ventilation system or in towns to improve the air quality.

Preferably, the carbon dioxide sorption unit comprises at least one chimney and at least one flow channel running transversely to the chimney, which is connected to the chimney at a region arranged below in installation position. The chimney preferably has the air outlet and the flow channel has the air inlet. Further preferably, the sorber device is arranged in flow direction between the flow channel and the chimney. The flow channel is preferably elongated and forms a region for the supplying of ambient air to the sorber device. The chimney is arranged downstream of the sorber device and directs the cleaned ambient air from the sorber device into the outside atmosphere.

The chimney can be arranged substantially perpendicularly to the flow channel. The air outlet and the air inlet preferably have a height offset with respect to one another. In other words, the air inlet and the air outlet are preferably vertically offset. The sorber device is preferably able to be flowed through by ambient air. It is advantageous here that through the configuration of the carbon dioxide sorption unit with the chimney and the flow channel a natural ventilation is realized, so that no electrically operated ventilator is necessary for air acceleration.

Nevertheless, it is possible that in a further embodiment a ventilator, in particular a blower, is provided, which delivers to the sorber device ambient air which is to be cleaned. This can be necessary for example on starting up the carbon dioxide sorption unit, in order to produce the natural chimney draught in the initial phase of the operation.

The at least one chimney can have a diameter of between 20 metres and 30 metres and a height of between 50 metres and 200 metres. The diameter of the chimney relates to the size of the air outlet. It is possible that the chimney has a larger diameter in the connection region of the flow channel than in the region of the air outlet. Preferably, the chimney has a diameter of 25 metres and a height of 100 metres. By such dimensions of the chimney, an optimized natural ventilation is made possible.

For example, with a diameter of 25 metres and a height of 100 metres of the chimney and with a first temperature of the ambient air outside the sorption unit of 40° C. and a second temperature of the ambient air within the sorption unit, in particular in the flow channel and/or in the chimney, an air ventilation or respectively an air throughflow quantity, in particular a cleaned ambient air quantity, with a number of forty chimneys of at least 1800 megatonnes is achieved per year.

The flow channel preferably has upwardly arranged area for solar ray absorption in installation position, in particular an at least partially dark-coloured area, in order to heat the ambient air, situated in the flow channel, by radiant heat. The flow channel is preferably arranged directly beneath the upwardly arranged area. The upwardly arranged area in installation position can be substantially black. The upwardly arranged area can be part of at least one a metal sheet. Alternatively, it is possible that the upwardly arranged area is part of at least one plate. Here, the natural ventilation is further improved for air movement between the flow channel and the chimney.

In a further embodiment, the upwardly arranged area is configured to be at least partially dark-coloured and at least partially light-coloured. Thereby an absorption and reflection of solar rays is made possible.

In a preferred embodiment, the upwardly arranged area is part of a planar plant region, on the longitudinal side of which several chimneys, in particular forty chimneys, are arranged in series, wherein beneath the upwardly arranged area a flow channel runs towards respectively at least one of the chimneys. The flow channels can be separated from one another respectively by a dividing wall. The flow channels preferably run in a parallel manner and are part of the planar plant region. Thereby, the carbon dioxide sorption unit has a space-saving and unified construction.

The planar plant region can be configured to be rectangular in top view. It is also possible that the planar plant region is configured to be circular in top view. The planar plant region preferably directly adjoins the further units of the plant, in order to keep the lines short.

In an embodiment, the planar plant region has at least one photovoltaic unit, which is arranged on the upwardly arranged area. The photovoltaic unit can be connected to the electrolysis unit for power supply. Alternatively or additionally, the photovoltaic unit can be connected to the carbon dioxide sorption unit for power supply. The photovoltaic unit can be configured as a photovoltaic field on the upwardly arranged area. Through the photovoltaic unit, the plant is able to be operated in an energy-autonomous manner. It is advantageous here that the plant is operated exclusively by power from solar energy and thus fossil fuels are entirely dispensed with for energy generation.

A method is also disclosed and claimed for operating a previously described plant for the utilization of the carbon dioxide content in atmospheric air for the production of a liquid fuel, in particular a plant for the reduction of the carbon dioxide content in atmospheric air and for the utilization of this carbon dioxide content at least partly for the production of a liquid fuel. In the method
a quantity of water is received by at least one electrolysis unit for oxygen production through at least one water supply line, and the received water quantity is decomposed by electrolysis into an oxygen partial quantity and a hydrogen partial quantity;
the hydrogen partial quantity is directed by at least one hydrogen transport device at least partially to a methanol synthesis unit;
ambient air of an outside atmosphere surrounding the plant is cleaned by at least one carbon dioxide sorption unit, wherein the ambient air is delivered through at least one air inlet to a downstream sorber device and subsequently a carbon dioxide quantity is extracted by the sorber device from the delivered ambient air; and
the carbon dioxide quantity is directed by at least one carbon dioxide transport device to the methanol synthesis unit (34),

wherein the oxygen partial quantity and the cleaned ambient air are emitted into the outside atmosphere, and the hydrogen partial quantity and the carbon dioxide quantity are converted to methanol in the methanol synthesis unit.

In this method, the plant is also supplied with power in a self-sufficient manner from one or more, particularly exclusively, regenerative energy sources.

Advantageous further developments and advantages of the method will emerge from the above description of the associated plant. The preferred further developments and advantages described in connection with the method according to Claim 17 apply accordingly also to the method according to Claim 23.

The invention is explained more closely below with further details with reference to the enclosed drawings. The illustrated embodiments represent examples as to how the plant according to the invention can be configured.

In these there are shown

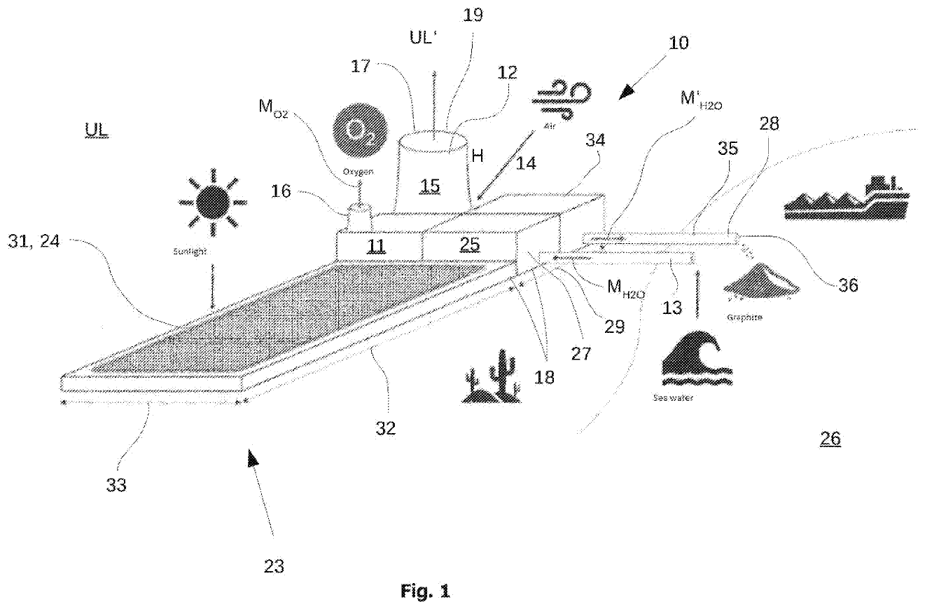

FIG. 1 a perspective view of a plant according to the invention for reduction of the carbon dioxide content in atmospheric air according to a preferred example embodiment;

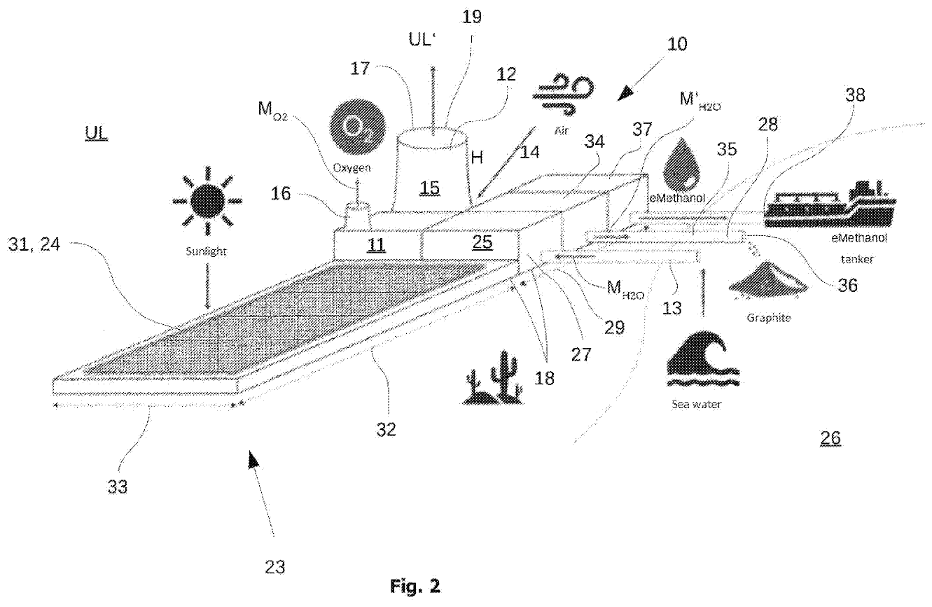

FIG. 2 a perspective view of a plant according to the invention for reduction of the carbon dioxide content in atmospheric air and for the production of a climate-neutral liquid fuel according to a preferred example embodiment;

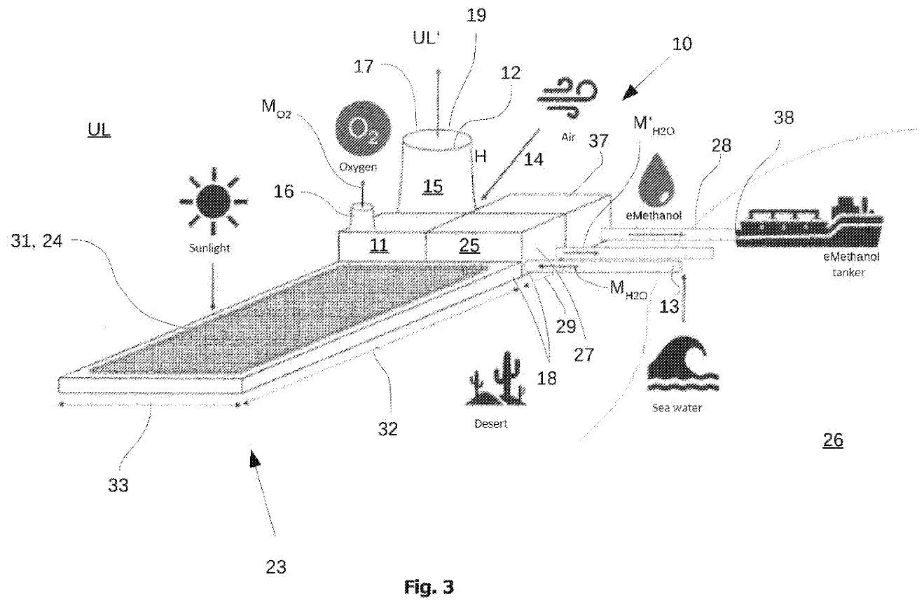

FIG. 3 a perspective view of a plant according to the invention for the production of a climate-neutral liquid fuel according to a preferred example embodiment; and

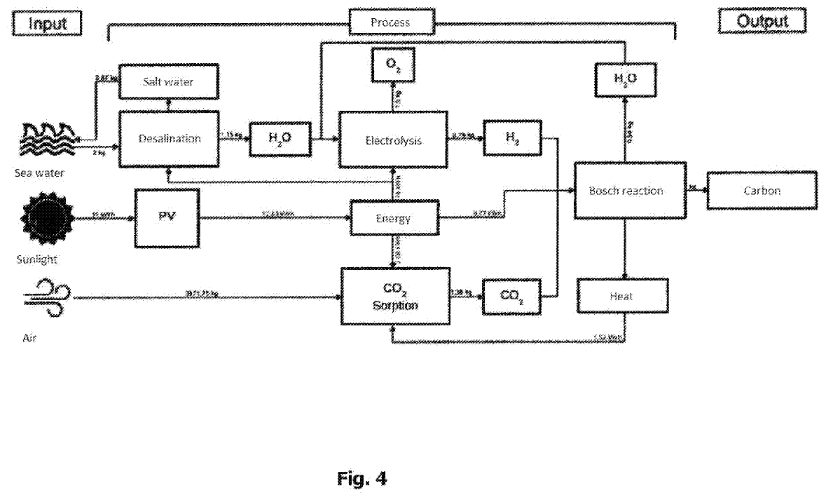

FIG. 4 a flow chart of a method according to the invention for operating the plants according to FIG. 1 or 2 according to a preferred example embodiment.

FIG. 1 shows a perspective view of a plant 10 for reduction of the carbon dioxide content in atmospheric air according to a preferred example embodiment. The plant 10 comprises an electrolysis unit 11 for oxygen production and a carbon dioxide sorption unit 12 for cleaning ambient air UL of an outside atmosphere surrounding the plant 10. The plant 10 further comprises a power generating unit 31 for the self-sufficient power supply of the plant 10, which is described more closely later.

The electrolysis unit 11 is configured to decompose a water quantity M_{H20} by electrolysis into an oxygen partial quantity M_{O2} and a hydrogen partial quantity. The electrolysis unit 11 thus forms a unit for water electrolysis. The electrolysis unit 11 is connected to a water supply line 13 to receive the water quantity M_{H2O}. As can be seen in FIG. 1, a pump unit 25 is arranged between the electrolysis unit 11 and the water supply line 13. The pump unit 25 has at least one pump for conveying water from a water reservoir 26. The water reservoir 26 can be a sea with sea water. Alternatively, the water reservoir 26 can be a lake with fresh water. It is also possible that the water supply line 13 is connected to a river, in order to remove fresh water for the water electrolysis. In the plant 10 shown in FIG. 1, the water supply line 13 is connected to a sea for the removal of sea water. The plant 10 is arranged near the coast, in order to keep short the distance to be overcome for water supply, in particular the water supply line 13.

The pump unit 5 is configured to convey sea water from the sea and to make it available to further plant parts or respectively units for further processing. In order to prepare the sea water for the electrolysis process by the electrolysis unit 11, the plant 10 has a sea water desalination unit 27. The sea water desalination unit 27 is connected to the pump unit 25 by at least one pipeline or is integrated into the pump unit 25. The sea water desalination unit 27 is adapted to separate a particular salt content from the conveyed sea water quantity M_{H2O}, so that after the desalination process by the sea water desalination unit 27 the sea water has a reduced salt content. The desalinated sea water quantity M_{H2O} corresponds to the water quantity M_{H2O} which is decomposed by the electrolysis unit 11 into an oxygen partial quantity M_{O2} and a hydrogen partial quantity. The electrolysis unit 11 is connected to the sea water desalination unit 27 by at least one pipeline. In order to convey the desalinated sea water from the sea water desalination unit 27 to the electrolysis unit 11, at least one further pump can be interposed.

As described above, the electrolysis unit 11 is designed to decompose the received water quantity M_{H2O} into a hydrogen partial quantity and an oxygen partial quantity M_{O2}. For emitting the produced oxygen partial quantity M_{O2}, the electrolysis unit 11 has an oxygen outlet 16, which opens into the outside atmosphere. It is possible that the electrolysis unit 11 has one or more oxygen outlets 16 for emitting the produced oxygen partial quantity M_{O2}.

The plant 10 has, furthermore, at least one hydrogen transport device, not illustrated, which is adapted to make available to a carbonation unit 34 for further processing the hydrogen partial quantity which is separated from the water quantity M_{H2O}. It is possible that, for this, the plant 10 has an intermediate hydrogen store, which is connected to the hydrogen transport device. After the electrolysis process, the hydrogen transport device directs the separated hydrogen partial quantity from the electrolysis unit 11 to the intermediate hydrogen store or to the carbonation unit 34 directly. Alternatively, it is possible that the hydrogen transport device delivers the hydrogen partial quantity to a further plant part which is not illustrated, in order to be further processed.

According to FIG. 1, the carbon dioxide sorption unit 12 has an air inlet 14 for the supply of the ambient air UL and a downstream sorber device 15. It is possible that the carbon dioxide sorption unit 12 has one or more air inlets 14. The sorber device 15 is connected to the air inlet 14. The sorber device 15 is adapted to extract a carbon dioxide quantity from the ambient air UL. The carbon dioxide sorption unit 12 has, furthermore, an air outlet 17. The air outlet 17 serves for the emission of the ambient air UL′ which has been cleaned of carbon dioxide. The air outlet 17 can be aligned in vertical direction upwards and/or part of a chimney 19.

In practice, the sorber device 15 is arranged between the air inlet 14 and the air outlet 17. In operation, the ambient air UL flows through the air inlet 14 to the sorber device 15, which separates, in particular filters, a particular carbon dioxide quantity from the air UL, wherein after the sorber device 15 the cleaned ambient air UL′ flows through the air outlet 17 into the outside atmosphere. Generally, it is possible that several air inlets 14, several sorber facilities 15 and several air outlets 17 are provided.

In practice, in FIG. 1 a single chimney 19 is illustrated with a height H of 200 metres, which by way of example shows the outer construction of the carbon dioxide sorption unit 12. As illustrated in FIG. 1, the air outlet 17 opens, like the oxygen outlet 16, into the outside atmosphere.

The plant 10 furthermore comprises a carbon dioxide transport device, which is configured to make available the carbon dioxide quantity, separated from the ambient air UL, to a carbon dioxide intermediate store and/or to the carbonation unit 34 of the plant 10 for further processing. Preferably, at least a portion of the hydrogen partial quantity and at least a portion of the carbon dioxide quantity are thus delivered to the carbonation unit 34, so that the extracted carbon dioxide quantity is processed with the separated hydrogen partial quantity to further intermediate and/or end products. In practice, at least a portion of the carbon dioxide quantity and at least a portion of the hydrogen partial quantity can be converted to water, carbon (graphite) and heat in a Bosch reaction, which is carried out in the carbonation unit (34), which is preferably configured as a Bosch reaction unit.

As shown in FIG. 1, the plant 10 has a planar plant region 23. The planar plant region 23 directly adjoins the electrolysis unit 11. On the planar plant region 23 a power generating unit 31 is arranged, which is a photovoltaic unit 24. The photovoltaic unit 24 is connected to the respective units of the plant 10 for power supply. The photovoltaic unit 24 is adapted such that the entire plant 10 is able to be operated in an energy-autonomous manner. This is be understood to mean that the electric current for operating the entire plant 10 is provided exclusively by solar energy by means of the photovoltaic unit 24. In other words, no fossil energy sources are used for the operation of the plant 10.

The planar plant region 23 has a longitudinal extent 32 of ca. 5000 metres and a transverse extent 33 of ca. 2000 metres. In other words, the planar plant region of the plant 10 is formed on an area of 10 square kilometres. The plant region shown in FIG. 1 containing the electrolysis unit 11 can have a partial longitudinal extent 29 of ca. two kilometres. Other partial longitudinal, longitudinal and transverse extents 29, 32, 33 are possible.

Proceeding from an overall area of the plant 10 of ca. twelve square kilometres, the plant 10 produces at least 580 metric tons of oxygen per hectare (0.01 square kilometres) per year. Compared to a conventional natural forest, which emits an annual oxygen quantity of 15 to 30 metric tons per hectare, the plant 10 has a 5 to 40 times higher oxygen output into the atmosphere. The plant 10 can therefore be designated as an artificial forest, which has a higher oxygen output rate than natural forest. In this respect, the plant according to the invention offers an approximately 30 times more efficient land utilization than the natural forest.

The sea water desalination unit 27 described above is connected to a water return line 28, through which a sea water quantity M′_{H2O} with increased salt content which is to be returned is returned into the sea. In practice, a particular salt content is extracted from the removed sea water quantity and is subsequently returned into the sea again with a portion of the removed sea water quantity as water quantity M′_{H2O} which is to be returned. Thereby, a water cycle is provided, which is harmless for nature.

The preferred installation site of the plant 10 is near to the coast of a sea. Particularly preferably, the plant 10 is constructed in a desert. The plant 10 according to FIG. 1 is a large power plant. The plant 10 has at least one mounting region 18, which is connected to a base of a building and/or of a structure. Generally it is possible that the electrolysis unit 11 and/or the carbon dioxide sorption unit 12 are arranged in a shared building or in separate buildings.

The power supply unit 31 preferably has a power store, which is not illustrated, which is adapted for power supply of the plant 10 in nighttime operation.

The plant 10 according to FIG. 2 is largely identical to the plant 10 according to FIG. 1 and differs only by a supplementation of the plant 10 by a methanol synthesis unit 37. The methanol synthesis unit 37 is connected to the electrolysis unit 11 or to a hydrogen intermediate store by a hydrogen transport device, and to the carbon dioxide sorption unit 12 by a carbon transport device. From the delivered hydrogen and carbon, the methanol synthesis unit 37 synthesizes methanol, which can be removed from the plant 10 via a methanol outlet 38. The methanol can be distributed worldwide to decentralized methanol delivery points by means of a fuel distribution system, which can comprise ships, in particular tankers, tank freight trains and/or tank trucks. The methanol delivery points can be filling stations at which the methanol is provided for the fueling of motor vehicles, aircraft, ships or locomotives.

Through a corresponding control of the method in the plant 10, an adjustment can be carried out as to which proportion of the carbon which is sorbed in the carbon dioxide sorption unit is used for the production of the liquid fuel methanol or for the production of graphite for storage in a carbon store. Initially, probably a ratio of 20% graphite and 80% methanol will be expedient, wherein the proportion of methanol is reduced successively in the further course, and the proportion of graphite is increased, when the requirement for methanol production falls in particular through the construction of further plant 10.

Generally, the plant illustrated in FIGS. 1 and 2 additionally use the carbonation unit 34, which is preferably a Bosch reaction unit. In particular, a reactor building can be provided, in which a reactor, preferably a fluidized bed reactor, is arranged, wherein in the reactor a Bosch reaction can take place. The carbonation unit 34 is preferably integrally incorporated into the plant 10, but can also be configured as a separate auxiliary plant. The carbonation unit 34 has a carbon outlet 36 which in the illustrated example embodiments is formed by the water return line 28 or opens into the latter. The required water quantity M_{H2O} removed from the water reservoir 26 for the electrolysis is not entirely split into hydrogen and oxygen in the plant 10. Rather, a residual quantity of water remains, which is returned into the water reservoir 26 as returned water quantity M′_{H2O} via the water return line 28. The graphite produced in the carbonation unit 34 is preferably also directed here into the water reservoir 26, which is preferably the sea. A cone of inert graphite, which can be additionally used as a reef, thus forms on the seabed and thus promotes the biodiversity in the sea.

In FIG. 3 a plant 10 is shown, that substantially is intended for the transition phase in which the production of a climate-neutral liquid fuel has priority. The plant 10 according to FIG. 3 corresponds substantially to the plant 10 according to FIG. 2, but dispenses with the carbonation unit 34. However, this can be retrofitted later. The plant 10 according to FIG. 3 thus serves exclusively for the production of a liquid fuel, in particular of methanol.

For the plant 10 illustrated in the drawings, it generally applies that the carbon dioxide can be removed from the air not only via the carbon dioxide sorption unit. Rather, it is also possible that the plant 10 has a carbon dioxide extraction unit which is connected to the water supply line 13 and extracts carbon dioxide from the removed water quantity M_{H2O}. The carbon dioxide extraction unit can be provided alternatively to the carbon dioxide sorption unit 12. However, it is preferred if the carbon dioxide extraction unit is provided additionally to the carbon dioxide sorption unit 12.

The method for operating the plant 10 according to FIG. 1 and/or according to FIG. 2 is described more closely in the following.

In a first method step, a water quantity M_{H2O} is received by means of the electrolysis unit 11 for oxygen production through the water supply line 13. Subsequently, the received water quantity M_{H2O} is decomposed into an oxygen partial quantity M_{O2} and a hydrogen partial quantity by an electrolysis process. The hydrogen partial quantity is made available by at least one hydrogen transport device to a carbonation unit 34 for further processing, wherein in the present example embodiment the carbonation unit 34 carries out a Bosch reaction.

In a second method step, ambient air UL of an outside atmosphere surrounding the plant 10 is cleaned by the carbon dioxide sorption unit 12. The ambient air UL is introduced, in particular drawn in, through several air inlets 14 into the flow channels 21, and is delivered to the downstream sorber facilities 15. Subsequently, the sorber facilities 15 extract a carbon dioxide quantity from the delivered ambient air UL. The carbon dioxide quantity is delivered to the Bosch reaction by the carbon dioxide transport device. Subsequently, after the breaking-down process, the obtained oxygen partial quantity M_{O2}, and the cleaned ambient air UL′ after the extraction of the carbon dioxide quantity, is emitted into the outside atmosphere. Thereby, the oxygen content in the air is increased and the CO₂ content in the air is reduced.

Furthermore, the hydrogen partial quantity together with the carbon dioxide quantity is converted by means of the Bosch reaction into water, carbon or respectively graphite, and heat, which is explained more closely in the following with the aid of the flow chart according to FIG. 4. The method described here, in particular the method shown in FIG. 4, is preferably carried out in one of the plant according to FIGS. 1 and 2.

In the method, sea water is desalinated, and the desalinated sea water is subsequently split by means of electrolysis into hydrogen and oxygen. The oxygen O₂ is emitted to the ambient air, in particular into the atmosphere, so that the oxygen content in the environment of the plant is increased. Parallel thereto, carbon dioxide CO₂ is collected from the ambient air UL, in particular the atmosphere, by means of a carbon dioxide sorption. The carbon dioxide or respectively the carbon dioxide quantity which is removed from the ambient air UL is directed, like the electrolytically produced hydrogen or respectively the hydrogen partial quantity, to the carbonation unit 34. In a Bosch reaction, which is carried out by means of a catalyst, such as for example iron, cobalt, nickel and/or ruthenium, 1 part pure carbon (graphite) and 2 parts water arise. The water is preferably directed back to the electrolysis, in order to reduce the consumption of sea water and the effort, connected therewith, for its desalination.

The carbon or respectively graphite can be subsequently delivered to a carbon store via the carbon transport device 35. The carbon store can be, for example, the water reservoir 26 or respectively the sea. As the graphite arising in the Bosch reaction has scarcely any to no impurities and is solidified in a rock-like manner, there are no objections to dumping the graphite in the sea.

The Bosch reaction preferably takes place at temperatures between 530° C. and 730° C., and particularly preferably in a fluidized bed reactor. With the use of a fluidized bed reactor, in particular iron granulate can be used as catalyst.

In the Bosch reaction, heat occurs as a product, in addition to water and graphite. This heat is utilized efficiently for the carbon dioxide sorption. Here, the heat can function as energy carrier for the carbon dioxide sorption, for example in order to promote the natural ventilation in the chimneys 19.

The energy required for the electrolysis, the carbon dioxide sorption and the Bosch reaction originates from regenerative energy sources, in practice from the photovoltaic unit 24, so that no additional production of carbon dioxide takes place here.

Through the method which is described here, it is consequently possible to remove carbon dioxide efficiently from the earth's atmosphere and to divide it into its components graphite and oxygen. The oxygen can be returned into the atmosphere, and the graphite can be stored permanently in a carbon store, for example in the sea.

By the invention, an improvement to atmospheric air quality is efficiently achieved.

### LIST OF REFERENCE NUMBERS

10 plant
11 electrolysis unit
12 carbon dioxide sorption unit
13 water supply line
14 air inlet
15 sorber device
16 oxygen outlet
17 air outlet
18 mounting region
19 chimney
23 planar plant region
24 photovoltaic unit
25 pump unit
26 water reservoir
27 sea water desalination unit
28 water return line
29 partial longitudinal extent
31 power generating unit
32 longitudinal extent
33 transverse extent
34 carbonation unit
35 carbon transport device
36 carbon outlet
37 methanol synthesis unit
38 methanol outlet
UL ambient air
UL′ cleaned ambient air
M_{H2O} removed water quantity
M′_{H2O} returned water quantity
M_{O2} oxygen partial quantity

1-23. (canceled)

24. A plant adapted for reduction of carbon dioxide content of atmospheric air comprising:
at least one electrolysis unit configured for oxygen production, the electrolysis unit connected to at least one water supply line to receive a water quantity, the electrolysis unit adapted to decompose the received water quantity by electrolysis into an oxygen partial quantity and a hydrogen partial quantity;
at least one hydrogen transport device adapted to connect the electrolysis unit to a carbonation unit for carbon synthesis;
at least one carbon dioxide sorption unit having at least one air inlet for supplying ambient air of an outside atmosphere surrounding the plant, the carbon dioxide sorption unit configured to extract a carbon dioxide quantity from the ambient air; and
at least one carbon dioxide transport device adapted to connect the carbon dioxide sorption unit to the carbonation unit,
wherein the electrolysis unit has at least one oxygen outlet for emission of the oxygen partial quantity, and the carbon dioxide sorption unit has at least one air outlet for emission of cleaned ambient air, wherein the oxygen outlet and the air outlet open into the outside atmosphere, wherein the carbonation unit has a carbon outlet for removal of carbon, and wherein at least one power generating unit is provided for self-sufficient power supply of the plant using one or more regenerative energy sources for power generation.

25. The plant according to claim 24, wherein the hydrogen transport device and the carbon dioxide transport device are additionally connected to a methanol synthesis unit for production of methanol, wherein the methanol synthesis unit has a methanol outlet for the removal of methanol.

26. The plant according to claim 24, wherein the power generating unit comprises one or more of:
a photovoltaic unit configured for conversion of solar energy into power,
a wind power unit for the conversion of wind energy into power,
a water power unit for the conversion of water energy into power, or
a thermal unit for the conversion of thermal energy into power.

27. The plant according to claim 24, wherein the carbonation unit is connected to the electrolysis unit by at least one water transport device.

28. The plant according to claim 24, wherein the carbonation unit is connected to a carbon store by a carbon transport device.

29. The plant according to claim 28, wherein the carbon transport device is formed at least partially by a water return line.

30. The plant according to claim 24, wherein the carbonation unit has a catalyst comprising one of iron, cobalt, nickel or ruthenium.

31. The plant according to claim 24, wherein at least one carbon dioxide extraction unit is connected to the water supply line for extraction of carbon dioxide from the water quantity.

32. A plant configured to use carbon dioxide content in atmospheric air for production of a liquid fuel comprising:
at least one electrolysis unit configured for oxygen production, the electrolysis unit connected to at least one water supply line to receive a water quantity, the electrolysis unit adapted to decompose a received water quantity by electrolysis into an oxygen partial quantity and a hydrogen partial quantity;
at least one hydrogen transport device, adapted to connect the electrolysis unit to a methanol synthesis unit for the production of methanol;
at least one carbon dioxide extraction unit connected to the water supply line for extraction of carbon dioxide from the water quantity;
at least one carbon dioxide sorption unit having at least one air inlet for supplying ambient air of an outside atmosphere surrounding the plant, the carbon dioxide sorption unit configured to extract a carbon dioxide quantity from the ambient air; and
at least one carbon dioxide transport device adapted to connect the carbon dioxide sorption unit to the methanol synthesis unit,
wherein the electrolysis unit has at least one oxygen outlet for emission of the oxygen partial quantity and the carbon dioxide sorption unit has at least one air outlet for emission of cleaned ambient air, wherein the oxygen outlet and the air outlet open into the outside atmosphere, wherein the methanol synthesis unit has a methanol outlet for removal of methanol, and wherein at least one power generating unit is provided for self-sufficient power supply of the plant using one or more regenerative energy sources for the power supply.

33. The plant according to claim 32, wherein the electrolysis unit has an output rate of an oxygen partial quantity per year of at least 700000 metric tons and the carbon dioxide sorption unit has an extraction rate of a carbon dioxide quantity per year of at least 400000 metric tons.

34. The plant according to claim 32, wherein the electrolysis unit is adapted to separate an oxygen partial quantity of at least 1.2 kg or a hydrogen partial quantity of at least 0.1 kg from a water quantity of at least 1.5 kg.

35. The plant according to claim 32, wherein the carbon dioxide sorption unit is adapted to extract a carbon dioxide quantity of at least 1.1 kg from an ambient air quantity of at least 3300 kg.

36. The plant according to claim 28, wherein the water supply line and the water return line are resistant to salt water for receiving salt water from a water reservoir or returning the salt water into the water reservoir.

37. The plant according to claim 32, wherein the water supply line has a desalination device.

38. The plant according to claim 24, wherein the carbonation unit comprises one of a Bosch reaction unit, a Kvaerner process unit, or a carbon dioxide plasma burner unit.

39. The plant according to claim 24, wherein at least one buffer store is provided for storing energy.

40. A method for operating a plant comprising:
receiving a water quantity by at least one electrolysis unit for oxygen production through at least one water supply line, and decomposing the received water quantity by electrolysis into an oxygen partial quantity and a hydrogen partial quantity;
directing the hydrogen partial quantity by at least one hydrogen transport device at least partially to a carbonation unit;
cleaning ambient air of an outside atmosphere surrounding the plant by at least one carbon dioxide sorption unit having at least one air inlet for delivery of the ambient air; and
directing the carbon dioxide quantity by at least one carbon dioxide transport device to the carbonation unit,
wherein the oxygen partial quantity and the cleaned ambient air are emitted into the outside atmosphere, and the hydrogen partial quantity and the carbon dioxide quantity are converted in the carbonation unit to water, carbon and heat, wherein the plant is supplied in a self-sufficient manner with power from one or more regenerative energy sources.

41. The method according to claim 40, wherein a portion of one of the hydrogen partial quantity or the carbon dioxide quantity is directed at least partially to a methanol synthesis unit for production of methanol.

42. The method according to claim 40, wherein the water is directed from the carbonation unit at least partially to the electrolysis unit and used for production of hydrogen.

43. The method according to claim 40, wherein the carbon is delivered to a carbon store for long-term storage.

44. The method according to claim 40, wherein the heat is directed from the carbonation unit to the carbon dioxide sorption unit and used as energy for carbon sorption.

45. The method according to claim 40, wherein a Bosch reaction takes place in the carbonation unit for production of carbon from hydrogen and carbon dioxide.

46. A method for operating a plant comprising:
receiving a water quantity by at least one electrolysis unit for oxygen production through at least one water supply line, and decomposing the received water quantity by electrolysis into an oxygen partial quantity and a hydrogen partial quantity;
directing the hydrogen partial quantity by at least one hydrogen transport device at least partially to a methanol synthesis unit;
cleaning ambient air of an outside atmosphere surrounding the plant by at least one carbon dioxide sorption unit having at least one air inlet for delivery of the ambient air; and
directing the carbon dioxide quantity to the methanol synthesis unit by at least one carbon dioxide transport device,
wherein the oxygen partial quantity and the cleaned ambient air are emitted into the outside atmosphere, and the hydrogen partial quantity and the carbon dioxide quantity are converted to methanol in the methanol synthesis unit, and wherein the plant is supplied with power in a self-sufficient manner from one or more regenerative energy sources.
